(19) United States
(12) Patent Application Publication
(10) Pub. No.: US 2012/0156244 A1
(43) Pub. Date: Jun. 21, 2012

(54) Nasal Compositions and Uses Thereof

(75) Inventor: Gerald Horn, Deerfield, IL (US)

(73) Assignee: ALPHA SYNERGY DEVELOPMENT INC., Dana Point, CA (US)

(21) Appl. No.: 13/406,321

(22) Filed: Feb. 27, 2012

Related U.S. Application Data
(63) Continuation-in-part of application No. 13/066,370, filed on Apr. 13, 2011.
Application No. 13/066,370 is a continuation-in-part of application No. 12/460,967, filed on Jul. 27, 2009.
(60) Provisional application No. 61/137,714, filed on Aug. 1, 2008.
(60) Provisional application No. 61/192,777, filed on Sep. 22, 2008.
(60) Provisional application No. 61/203,120, filed on Dec. 18, 2008.
(60) Provisional application No. 61/207,481, filed on Feb. 12, 2009.

(51) Int. Cl. A61K 31/502; A61K 31/165; A61K 31/4164; A61K 31/4168; A61K 39/08; A61K 31/407; A61K 31/4045; A61K 31/48; A61K 31/405; A61K 31/192; A61K 31/4706; A61K 38/28; A61K 31/573; A61P 11/02; A61P 25/00; A61P 37/08; A61P 1/04; A61P 27/16; A61P 25/06; A61P 25/28; A61P 25/24; A61P 3/10; A61P 11/06; A61P 9/10; A61P 11/00; A61P 11/08; A61K 31/197

(52) U.S. Cl. 424/239.1; 514/249; 514/567; 514/617; 514/400; 514/396; 514/392; 514/398; 514/413; 514/415; 514/250; 514/420; 514/570; 514/569; 514/313; 514/6.5; 514/171

(57) ABSTRACT

Pharmaceutical compositions for the treatment of nasal congestion, wherein the pharmaceutical compositions comprise low concentrations of a super-selective subclass of selective α-2 adrenergic receptor agonists. Methods of using the compositions for the treatment of nasal congestion, cerebrovascular disease or systemic conditions, and as delivery vehicles to deliver other active agents to treat systemic or cerebrovascular diseases or conditions.

### CROSS-REFERENCE TO RELATED APPLICATIONS

This application is a continuation-in-part of U.S. patent application Ser. No. 13/066,370 filed Apr. 13, 2011, which is a continuation-in-part of U.S. patent application Ser. No. 12/460,967 filed Jul. 27, 2009, which claims a priority of U.S. Provisional Application Ser. No. 61/137,714, filed on Aug. 1, 2008; 61/192,777, filed on Sep. 22, 2008; 61/203,120, filed on Dec. 18, 2008; and 61/207,481 filed on Feb. 12, 2009.

This application also claims a priority of U.S. patent application Ser. No. 12/798,925 filed Apr. 14, 2010, which claims a priority of U.S. Provisional Application Ser. No. 61/287,518 filed on Dec. 17, 2009, and which is a continuation-in-part of U.S. patent application Ser. No. 12/460,970 filed Jul. 27, 2009, which claims a priority of U.S. Provisional Application Ser. Nos. 61/137,714, filed on Aug. 1, 2008; 61/192,777, filed on Sep. 22, 2008; 61/203,120, filed on Dec. 18, 2008; and 61/207,481 filed on Feb. 12, 2009.

This application also claims a priority of U.S. patent application Ser. No. 12/928,761 filed Dec. 17, 2010, which claims a priority of U.S. Provisional Application No. 61/287,533 filed Dec. 17, 2009.

The contents of the above-mentioned applications are hereby incorporated by reference in their entirety.

### BACKGROUND OF THE INVENTION

Adrenergic receptors mediate physiological responses to the catecholamines, norephinephrine and epinephrine, and are members of the superfamily of G protein-coupled receptors having seven transmembrane domains. These receptors, which are divided pharmacologically into alpha-1 (α-1), alpha-2 (α-2) and β-adrenergic receptor types, are involved in diverse physiological functions including functions of the cardiovascular and central nervous systems. The α-adrenergic receptors mediate excitatory and inhibitory functions: α-1 adrenergic receptors are typically excitatory post-synaptic receptors which generally mediate responses in an effector organ, while α-2 adrenergic receptors are located postsynaptically as well as presynaptically, where they inhibit release of neurotransmitters. The α-adrenergic receptors also mediate vascular constriction. Agonists of α-2 adrenergic receptors are currently used for the treatment of hypertension, glaucoma, spasticity, and attention-deficit disorder, in the suppression of opiate withdrawal, as adjuncts to general anesthesia and in the treatment of cancer pain.

α-2 adrenergic receptors are present in various bodily organs, including eyes and nose. It is believed that they play a role in nasal congestion, among many other diseases.

α-2 adrenergic receptors are presently classified into three subtypes based on their pharmacological and molecular characterization: α-2A/D (α-2A in human and α-2D in rat); α-2B; and α-2C (Bylund et al., Pharmacol. Rev. 46:121-136 (1994); and Hein and Kobilka, Neuropharmacol. 34:357-366 (1995)). The α-2A, α-2B, and α-2C subtypes appear to regulate arterial and/or venular contraction in some vascular beds, and the α-2A and α-2C subtypes mediate feedback inhibition of norepinephrine release from sympathetic nerve endings.

Many compounds having selective α-2 agonist activity are known and include brimonidine (which has been used for lowering intraocular pressure in patients with open-angle glaucoma or ocular hypertension), guanfacine (which has been used to control high blood pressure), dexmedetomidine (which has been used as a sedative, analgesic, sympatholytic and anxiolytic), and methyl dopa (which has been used as a centrally-acting adrenergic antihypertensive).

Nasal conditions, such as nasal congestion, cause inconveniences and sufferings to many patients. The use of conventional decongestant nasal sprays cause rebound congestion often lasting 24 hours or longer which typically results after using these sprays for more than three consecutive days, and often after even a single day use. In addition, continued use of conventional nasal decongestants (such as Afrin®, Merck & Co; Dristan®, Pfizer; and many others) may result in chronic and long term inflammatory pathological conditions. They frequently result as a patient attempts to reverse the rebound congestion with more and more frequent use of the conventional nasal decongestant. Phenylephrine, a strong α-1 agonist, and oxymetazoline, a strong α-1 agonist with some α-2 agonist activity, are powerful nasal decongestants. However, they are associated with numerous side effects from repeat use. Rhinitis medicamentosa is one such result of inflammatory ischemic changes from such patterns of use, ultimately resulting in a total nasal blockage which may not be relieved by simply stopping the medication. It may take days, weeks, months, or even medical or surgical intervention to treat rhinitis medicamentosa. It is currently estimated that 10 million U.S. alone suffer from rhinitis medicamentosa.

It is a long held dogma of prior art that all topical a-agonists when used nasally induce vasoconstriction, and as a result, cause ischemia. Thus, it is thought that all topical a- agonists, when repeatedly topically applied to mucosal surfaces, result in rebound hyperemia and/or congestion, tachyphylaxis, and chronic ischemic inflammatory change, such as rhinitis medicamentosa.

It is well understood that allergic rhinitis in particular, and nasal congestion in general, are common disorders, affecting over 40 million individuals in the U.S. alone with long term and/or chronic treatment need, Some estimate that over $5 billion is spent annually on medications to relieve nasal obstruction, an additional $60 million on surgical remedies, and another $10 billion on the treatment of associated disorders. (Kimmelman C P. The problem of nasal obstruction. Otolaryngol Clin North Am 1989; 22:253-64). Many conditions are associated with nasal congestion and allergic rhinitis, including but not limited to asthma, other upper respiratory conditions, including bronchitis, sinusitis, gastroesophageal reflux, sleep apnea, ear infections, and migraine headaches.

Many conditions are known to increase in frequency during allergy season, particularly migraine headache. Experts have suggested that aggressive treatment of allergic rhinitis may help treat and prevent headaches in those people who appear to have allergic triggers to their migraines (Ku M, Silverman B, Pfifti N et al. Prevalence of Migraine Headaches in Patients with Allergic Rhinitis. Ann Allergy Asthma Immunol. 2006; 97: 226-30).

It is believed that many perceived sinus headaches are more accurately diagnosed as migraine headaches, and that allergic rhinitis is an associated condition. Patients with a high incidence of positive allergy tests have been found to be predisposed to migraine headaches, and 34% of patients with allergic rhinitis have migraine headaches vs. 4% in those without allergic rhinitis. Thus, allergic rhinitis may increase the frequency of migraine attacks.

Treatments of migraine headaches have been rendered difficult by the need for injections of several effective treatments, including narcotic injections (ketorolac tromethamine), Botox® (Allergan, Inc.), sumatriptan, ergotamines (dihydroergotamine), antihistamines, and other immunotherapeutic drugs. Thus, there is a need for new compositions and methods that would be useful for treatment of nasal conditions, including but not limited to nasal congestion, as well as systemic conditions. It would be especially desired to arrive at compositions and methods which provide long lasting relief with no or only transient rebound (i.e., less than 2 hrs), allowing daily administration for direct nasal benefit, direct central nervous system (CNS) benefit, other systemic benefit, and/or administration of a second drug.

There is also a need for new delivery vehicles for medications useful for the treatment of various diseases, whereby said medications can be administered through the nasal route to both relieve nasal congestion on a chronic basis and allow for a nasal route of drug administration on a daily or frequent basis without significant rebound congestion and/or rhinitis medicamentosa. These delivery vehicles would allow for a more uniform nasal delivery of drug, improved distribution to upper turbinates where greater CNS diffusion may occur, and improved consistency of delivered dose per use and from patient to patient, regardless of baseline congestion.

### SUMMARY OF THE PRESENT INVENTION

The present invention provides pharmaceutical compositions which can be used to treat nasal congestion by delivering low concentrations of a subgroup of selective α-2 adrenergic receptor agonists with only transient (i.e., lasting less than one or two hours), or no rebound congestion with repeated use, and without induction of rhinitis medicamentosa. The invention is based, in part, on a surprising discovery that while α-2 agonists are held to be less powerful vasoconstrictors and decongestants than α-1 agonists, the provided super-selective subclass of α-2 agonists at low concentrations produces comparable or greater nasal decongestion than α-1 and general a-agonists The provided compositions can be in the form of a nasal spray or a topical drop (i.e., a liquid drop).

In other embodiments, the provided compositions can be used as delivery vehicles to deliver medications which currently require injections or other routes of administration.

In one embodiment, the present invention provides a pharmaceutical composition comprising a highly selective α-2 adrenergic receptor agonist having a binding affinity of 900 fold or greater, and preferably 950 fold for α-2 over α-1 adrenergic receptors, or a pharmaceutically acceptable salt thereof, wherein said α-2 adrenergic receptor agonist is present at a concentration from between about 0.001% and about 0.075% weight by volume, and preferably 0.005% to 0.05% weight by volume.

In a preferred embodiment, the compositions of the invention comprise a mucoadhesive additive.

In another embodiment, the invention provides a method of delivering an active agent for the treatment of a systemic or cerebrovascular disease or condition comprising administering to a patient in need thereof the pharmaceutical composition of the invention, wherein said nasal spray composition further comprises the active agent.

Diseases and conditions that can be treated with the compositions and methods of the present invention include, but are not limited to, allergies; allergic rhinitis; disseminated intravascular coagulation; allergic shock; septic shock; gastro esophageal reflux; ear infection; sinusitis; nasal congestion; migraines; headaches; cervical dystonia; blepharospasm; spasticity; Alzheimer's disease, attention deficit disorder (ADD); depression, memory loss; sleep apnea; diabetes; asthma; transient ischemic cerebrovascular ischemic attacks (TIA's); cerebrovascular accident; degenerative cerebral disorder; pneumonia; acute respiratory distress syndrome (ARDS); acute lung injury (ALI); and infantile bronchiolitis.

### DETAILED DESCRIPTION OF THE INVENTION

### Definitions

For purposes of the present invention, the terms below are defined as follows.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™ (Allergan, Inc.), and UK14304.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

The term “nasal condition” refers to any disease, disorder, or condition which affects and/or involves the nose. This term includes, but is not limited to, such conditions as nasal congestion, diseases and/or conditions associated with swollen nasal turbinates, all types of rhinitis including but not limited to vasomotor rhinitis and allergic rhinitis, sleep apnea, acute or chronic sinusitis, nasal polyposis, and any disease and/or condition associated with nasal discharge.

The term “substantial enlargement of nasal turbinates” refers to a significant enlargement of nasal turbinates, for example, more than about 50% compared to the baseline level of the patient so that it negatively affects the patient's breathing.

### Embodiments of the Invention

The present invention provides compositions and methods which utilize low concentrations of a super-selective subclass of selective α-2 adrenergic receptor agonists for a variety of applications, including at least: 1) treatment of nasal congestion, 2) CNS or systemic delivery system for the selected α-2 agonists, and 3) a nasal delivery system for other active ingredients to treat a variety of diseases and conditions. The different applications of the invention are described in a greater detail below.

### Compositions and Methods for the Treatment of Nasal Decongestion and Other Conditions

In one embodiment, the present invention provides pharmaceutical compositions which can be used to treat nasal congestion by delivering low concentrations of 0.075% or less, more preferably 0.05% or less, and still more preferably of 0.035% or less of a super-selective subclass of highly selective α-2 adrenergic receptor agonists of 900 fold or greater, and still more preferably of 950 fold or greater α-2 to α-1 selectivity, with highly effective decongestant activity equal to or greater than that of phenylephrine and/or oxymetazoline (two commercially used alpha-agonists for topical nasal decongestion), and transient (i.e., typically lasting less than one or two hours), if any, rebound congestion without rhinitis medicamentosa.

It is an unexpected discovery of the present invention that contrary to a long held belief that all alpha-agonists cause rebound congestion (or rebound) due to vasoconstrictive ischemia, the cause of rebound congestion is the degree of α-1 agonist recruitment. Thus, a super selective subclass of α-2 agonists can reduce or eliminate this rebound. It has been also surprisingly found that this discovered subclass of highly selective α-2 agonists exhibits peak nasal decongestant effectiveness at a lower than previously believed concentration range for selective α-2 agonists, with equal to or greater effectiveness than that for α-1 agonists on single use. In preferred embodiments of the invention, the concentration of α-2 agonists is 0.075% or less, and in more preferred embodiments 0.035% or less, wherein all concentration units are weight by volume, unless otherwise noted. It is believed that the provided compositions produce a more effective nasal decongestant activity at these lower concentrations than the decongestant activity of selective α-2 agonists at higher concentrations (for example, than the activity of BHT 920 (6-allyl-2-amino-5,6,7,8-tetrahydro-4H-thiazolo-[4,5-d]azepine) at 1%. (Corboz et al, Pulmonary Pharmacology & Therapeutics 21 (2008) 449-454; Mechanism of decongestant activity of α-2-adrenoceptor agonists)).

Further, the provided compositions retain their effectiveness even when used repeatedly, and further, they result in only transient (<2 hrs) or no rebound, and with greatly diminished or eliminated risk of rhinitis medicamentosa. This is especially surprising and unexpected because a strong decongestant activity of the compositions of the invention would have been expected to result in greater rebound congestion with repeat use. The finding of minimal to absent rebound and rhinitis medicamentosa with repeat use of the present invention is, therefore, contrary to decades old unchallenged teachings of the prior art.

The present invention thereby provides a means to optimize α-2 nasal decongestion effectiveness for a particular subclass of α-2 agonists of defined “super” selectivity of 900:1 or greater, with surprisingly potent nasal decongestion and lack of ischemia with repeated use. Other α-2 agonists with similar profiles that may be considered for preferred embodiments based on these discoveries include fadolmidine, dexmedetomidine, guanfacine and guanabenz. Other selective α-2 agonists may be readily synthesized to demonstrate similar or greater α-2 selectivity. Alpha-2a, -2b, or -2c receptor selectivity is sufficient for purposes of the present invention.

Accordingly, the present invention, which provides nasal decongestant compositions for topical delivery (via either spray or drop) which cause minimal and transient (typically limited to at most a few hours, even with repeat use) or no rebound nasal congestion, has a variety of therapeutic benefits, such as:
1) providing lasting daily relief via a single daily application;
2) providing a safe alternative to conventional nasal decongestants;
3) providing a treatment to the estimated 40 million seasonal or chronic allergic rhinitis sufferers;
4) providing a treatment for sleep apnea, by reducing mucosal thickening, reducing peak nasal inflow resistance, and decreasing nasal airflow resistance;
5) providing a weaning treatment and/or a future alternative for the nearly 10 million people in the U.S. believed to suffer from rhinitis medicamentosa;
6) providing a novel nasal delivery for daily or frequent CNS distribution vs. oral, intravenous (IM), intramuscular (IM), or ocular routes; and
7) in some embodiments, allowing a frequent use of the provided formulations (such as daily, or even twice daily or occasionally thrice daily), particularly for patients who suffer from both nasal congestion and sleep apnea, allowing them to achieve a nearly round the clock decongestion.

In addition, generalized headaches may include various pathologic triggers and manifestations that variably include components of nasal allergy and/or migraine headache, such as vessel dilation, and that may for these or other reasons respond to triggering CNS α-2 receptors. Thus, generalized headaches, cluster headaches, and sinus headaches may be treatable to a useful degree by direct administration via nasal delivery of an α-2 agonist.

Thus, in one embodiment, the present invention provides compositions for nasal decongestion comprising a selective α-2 adrenergic receptor agonist having a binding affinity of 900 fold or greater for α-2 over α-1 adrenergic receptors, or a pharmaceutically acceptable salt thereof, wherein said α-2 adrenergic receptor agonist is is present at a concentration from between about 0.001% and about 0.075% weight by volume and more preferably 0.005% to 0.05% weight by volume.

In one embodiment, the compositions of the invention are in the form of a nasal spray. In another embodiment, the compositions of the invention are in the form of a topical drop, i.e. liquid.

In preferred embodiments, the selective α-2 adrenergic receptor agonist is selected from the group consisting of brimonidine, alpha methyl dopa, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yl)imino]indazole, and mixtures of these compounds.

In the most preferred embodiment, the selective α-2 adrenergic receptor agonist is brimonidine.

Preferably, the concentration of the selective α-2 adrenergic receptor agonist is between about 0.01% and about 0.075% weight by volume, more preferably, between about 0.015% and about 0.05%, and even more preferably between about 0.020% and 0.035%.

In preferred embodiments, the compositions of the invention include one or more mucoadhesive additives. In a preferred embodiment, the mucoadhesive or combination of mucoadhesive additives are selected from the group consisting of carboxymethylcellulose (CMC), hydroxypropylcellulose (HPMC) or other cellulose derivatives, carbomers and poloxamers. Preferably, the mucoadhesive additive is selected from the group consisting of a carbomer and a poloxamer or either in combination with a cellulose derivative like CMC or HPMC. Preferably, the concentration of poloxamer is between about 0.5% and about 20%; more preferably, between about 5% and about 15%; and even more preferably, between about 8% and about 12%. Preferably, the concentration of carbomer is between about 0.05% and 0.5% and more preferably 0.1% to 0.4%. Preferably, the concentration of a cellulose derivative is between 0.1% and 0.5%, and more preferably about 0.3%. In some preferred embodiments, additional inactive ingredients may be added, such as polyvinyl alcohol (PVA), chitosan, chondroitin sulfate, xanthan or other gum, chondroitin sulfate, other mucoadhesives and/or viscosity enhancers, or polyethylene glycol (PEG).

Preferred poloxamers include, but are not limited to, Poloxamer 407 (or its trade name Pluronic® F127 (BASF Corporation)), which can be used with or without a buffer.

Preferred carbomers include Carbopol 954 (Lubrizol Corporation) which can be used with or without a buffer.

The goal of an effective nasal decongestant is duration of efficacy of several hours, preferably four hours or more, and more preferably 6 hours or more.

Formulations with poloxamer concentrations of 2% to 20%, and brimonidine concentration of 0.035% or less offer preferred embodiments with the optimal combination of quick onset, minimal to no dryness, no sedation, and longer duration of effect.

In another preferred embodiment, the compositions of the invention incorporate two or more mucoadhesives, preferably any combination of two from the group consisting of poloxamers, carbomers, methylcellulose and the derivatives thereof, particularly, poloxamer concentrations of about 8% to 16%, hydroxypropylmethyl cellulose (HPMC) concentrations of about 0.1% to 0.5%., brimonidine concentrations of about 0.010% to 0.020%, and with or without polyvinyl alcohol of about 0.1% to about 0.5%.

The formulations of the invention, along with possible additional ingredients, preferred concentrations of active and inactive ingredients, etc, are described in a greater detail in the section of this application titled “Preferred Formulations of the Invention.”

The invention also provides a method of treating nasal congestion comprising administering to a patient in need thereof a pharmaceutical composition of the present invention.

In a preferred embodiment, the invention provides a method of treating nasal congestion comprising administering to a patient in need thereof a nasal spray composition comprising a selective α-2 adrenergic receptor agonist having a binding affinity of 950 fold or greater for α-2 over α-1 adrenergic receptors, or a pharmaceutically acceptable salt thereof, wherein said α-2 adrenergic receptor agonist is present at a concentration from between about 0.001% and about 0.075% weight by volume.

In addition, it was surprisingly and unexpectedly found that co-administration of the compositions of the present invention through nasal route and topical ophthalmic route greatly enhances the duration of the nasal decongestion effect of the compositions of the present invention administered nasally.

Accordingly, in one embodiment, the invention provides a method of treatment of nasal congestion comprising nasally administering to a patient in need thereof a composition of the present invention and further comprising topically administering to said patient a composition of the present invention as an eye drop.

### Compositions and Methods for Nasal Delivery of Medications

In other embodiments, the present invention provides compositions and methods for the use as a nasal delivery system to deliver the active ingredient and/or other active ingredients which currently require injections or other routes of administration to treat a variety of diseases and conditions. Therefore, the provided nasal spray compositions result in their more consistent delivery locally or beyond, and can be used as delivery vehicles to deliver other medications.

α-2 agonists, particularly brimonidine and dexmedetomidine, have multiple inotropic effects (i.e., the force or energy of muscular contractions) on cellular mechanisms that include systemic, vascular, direct neuroprotective, and indirect neuroprotective therapeutic benefits. At modestly increased concentrations, sedation, due primarily to α-2 receptors at the locus ceraeulus of the brainstem, may occur. While not wishing to be held to a particular theory, some of the specific α-2 agonist mediated inotropic effects include but are not limited to:
i) increased extracellular signal-regulated kinase (ERK) phosphorylation;
ii) reduced neuronal ischemia and cell death secondary to vascular infarction;
iii) reduced inflammation and vascular leakage with less polymorphonuclear (PMN) cells recruitment and cytokine induced postcapillary venular leakage;
iv) anti-inflammatory suppression of cytokines TNF-a, IL-6, and others;
vi) suppression of systemic norepinephrine and catecholamine levels and induced ischemia;
vii) anxiolysis and sedation and/or bypass of the blood brain barrier;
viii) multiple neuroprotective mechanisms, including increased glutamate synthetase levels with breakdown of excitotoxic glutamate; excitatory amino acid—NMDA pathway neuronal apoptosis suppression; reduction in apoptotic protein synthesis and increase in anti-apoptotic protein neuronal synthesis; decreased mictochondrial lysis and reduction in mitochondrial cytochrome P450; reduced infarct ischemia in cerebral, optic nerve, and retinal ganglion cell models; neuroprotective benefits with long term use and suppression of visual field loss in glaucoma.

However, before the present invention, delivery of α-2 agonists for the treatment of systemic or CNS diseases or conditions required oral, intramuscular, intra-peritoneal, or intravenous administration, typically resulting in high levels of sedation or other side effects and inconvenience. The delivery of α-2 agonists through nasal administration was problematic due to the high incidence and variability of the nasal congestion in a general population, limiting consistency and predictability of dosing

The present invention provides means of nasal decongestion without rebound congestion, allowing daily administration of α-2 agonists (optionally, with additional active agents) through nasal delivery so that the drugs can be absorbed systemically or diffuse into the CNS particularly near the upper turbinates via the nasal cribiform plate, thus bypassing the blood brain barrier. The present invention allows for an even more frequent or daily nasal administration of α-2 agonists or additional drugs, while the conventional nasal decongestants may cause rebound congestion and/or rhinitis medicamentosa with repeated use, and therefore, can typically only be used once a day for a period of about three days.

Addition of other drugs to the provided formulations of α-2 agonists for nasal administration, including but not limited to ketamine, such as for example to reduce N-Methyl-D-aspartate (NMDA) receptor excitotoxicity may more effectively treat asthma or more particularly, shock (anaphylactic, septic, smoke inhalation, etc.), and may further improve successful treatment of these and other conditions by safer and or more convenient delivery.

It is a further discovery of the present invention that rhinitis medicamentosa and rebound nasal congestion are α-1 derived receptor triggered events. Because the formulations of the present invention do not include α-1 agonists, and are formulated with highly selective α-2 agonists at very low concentrations, the formulations of the present invention have only negligible α-1 activity.

It is a discovery of the present invention that rebound is an event extremely sensitive to α-1 receptor recruitment, and that even selective α-2 agonists, when less selective than the α-2 subclass of the present invention may be insufficiently selective and/or at relatively too high concentrations, and may recruit sufficient α-1 receptors in sufficiently high numbers to cause rebound congestion. Not wishing to be held to any particular theory, this may be due to profound mucosal and vascular sensitivity to ischemic consequence of stimulation of α-1 receptors they induce via their relative α-2/α-1 selectivity, and co-dependent concentration driven increased α-1 receptor recruitment.

The provided compositions can be used for the treatment of allergic rhinitis and other causes of nasal congestion, and/or delivering other active drugs through either nasal spray or topical drop applications virtually free of α-1 effects.

Other conditions, such as migraine, may be more effectively treated with the present invention, as the nasal congestion often associated with migraines can be reduced and/or eliminated improving comfort. Further, treatment of the allergic rhinitis may reduce the migraine trigger and frequency and/or severity of migraine attacks. Still further, the delivery of the active ingredient super-selective α-2 agonist to the CNS directly may cause desired CNS reduction in vascular dilatation and or cause alpha 2 induced vasoconstriction and prevent, reduce, or reverse some or all forms of migraine headaches and their sequelae via triggering CNS α-2 receptors. An improved direct CNS drug absorption may be achieved via a single spray per nostril, or increased after onset of decongestant effect via administration of one or more additional sprays per nostril, particularly with inhalation after spray to achieve maximal superior turbinate distribution.

Botox injections for the treatment of migraine headaches typically involve about 155 units of Botox® (Allergan) being injected into 31 sites. Intranasal administration via spray or drop with the present invention combines direct systemic and/or CNS benefits of super-selective α-2 agonist absorption along with local rebound-free decongestion with potential absorption of one of several currently injected medications for treatment of migraine. Addition of any second medication, including but not limited to migraine treatment medications for intranasal delivery, may be rendered more effective with the addition of absorption enhancers well known to experts in the art, including but not limited to beta-cyclodextrin, dimethyl-beta-cyclodextrin, and/or its derivatives; surfactants, including but not limited to laureth-25; mucolytic agents, including but not limited to N-acetyl-l-cysteine (NAC); powder formulations, including but not limited to cellulose derivatives such as microcrystalline cellulose and ethyl cellulose, sodium glycholate and derivatives.

Further, the compositions and methods of the invention may be used to treat shock, which results from microvascular leakage with loss of intravascular volume. Current treatments of shock with vasoconstrictors often cause induced ischemic consequence, inflammatory reaction, and systemic rebound dilatation and leakage. It is believed that shock may be treated via systemic absorption via nasal administration of α-2 agonists of the present invention. Either a single or multiple sprays may be used to generate desired systemic levels of the super-selective subclass of α-2 agonists of the present invention. The administration of these α-2 agonists may take place prior to, concurrently, after, or instead of administration of commonly used vasoconstrictors, such as dopamine, epinephrine, norepinephrine and others. Therefore, many forms of shock, including but not limited to, disseminated intravascular coagulation (DIC), anaphylactic shock, septic shock, acute lung injury (ALI) and acute respiratory distress syndrome (ARDS) and other similar pathologic conditions may be more effectively treated with the compositions and methods of the present invention, and may be aided by selection of dexmedetomidine as the super-selective α-2 agonist and/or by the addition of a second drug, such as ketamine.

In addition, the present invention can be used for the treatment of allergies. In one embodiment, the treatment is through allergen desensitization which until the present invention requires repeated injection of low dose allergens. Previously, nasal delivery of allergens was difficult or even impossible because of rebound nasal congestion triggered by nasal administration of an allergen. However, because the formulations of the present invention provide nasal decongestion virtually without rebound effect, the present invention allows replacing injection of one or several allergens by their nasal delivery, which may be repeated daily, and eases self-administration of the allergens.

Further, the invention facilitates both cerebral non-blood brain barrier delivery and systemic delivery. In prior art, nasal delivery of medications was limited by mucosal thickness (normally, between 2-4 mm), turbinate patency upon which the distribution of drug is predicated, and the ciliary and vascular leakage of the drug. The present invention facilitates nasal drug delivery in at least the following ways:
1) by resulting in a thinner mucosa, allowing the drug to penetrate into the rich vascular plexus underneath the mucosa more quickly with less leakage of the drug;
2) by shrinking the mucosa, there is a greater access to the cribiform plate, through which the nasal nerves (and their microcirculation) exit from the brain into the nose;
3) by reducing vascular leakage, allowing improved drug entrance into the vascular plexus;
4) by reducing ciliary clearance, promoting greater drug retention;
5) by improving mucoadherence, resulting in greater drug retention; and
6) by allowing for quick onset of drug action (seconds to minutes), so that an optional second spray can achieve a much greater distribution throughout the nasal turbinates, and a much greater penetration into the upper nasal turbinates, facilitating both systemic and cerebral blood-brain barrier bypass and absorption.

When used as delivery vehicles, the provided nasal spray compositions reduce mechanical barriers to upper aspects of nose and lower aspects of brain and/or systemic absorption by providing nasal mucosal decongestion, which allows for a more accurate and effective lower concentration bolus of medication to be applied. Further, because the nasal spray compositions result in little, if any, rebound congestion, which when found is typically reversed within two hours vs. many days as in prior art, they can be applied daily without experiencing rebound, or even more than one time per day for long periods of time, thus providing lasting nasal decongestant effect. Another advantage of using the provided compositions is that they can limit bleeding when administered prior to an injection to the nasal mucosa. Further, in some embodiments, the provided nasal compositions can be combined with an allergen to provide desensitization effect for the treatment of allergies.

Further, the invention allows for delivery of brimonidine via diffusion through nasal sensory nerves and/or arteries/arterioles in the upper turbinates, particularly the region of the cribiform plate directly into the brain without the need to cross the blood brain barrier, as via systemic administration. Many known central nervous system benefits have been found as a result of systemic, oral, intravenous, and/or intraperitoneal administration of α-2 agonists.

Some of the medications which can be delivered via nasal administration with the use of the provided compositions include allergy medications, botulinum toxin, including but not limited to onabotulinumtoxinA (Botox®, Allergan, Inc.); ketorolac tromethamine (Toradol®, Roche Pharmaceuticals); sumatriptan (Imitrex®, GlaxoSmithKline Pharmaceuticals); ergotamines, including but not limited to dihydroergotamine (Migranal®, Valeant Pharmaceuticals); sulfonylureas (oral hypoglycemic); immunotherapeutics; IgE suppressors, including but not limited to antihistamines; non-steroidal anti-inflammatory drugs (NSAIDs) including but not limited to indomethacin, ibuprofen, sulindac sulfide, meclofenamic acid, and flurbiprofen; hydroxycholoroquine, selective COX-1 or COX-2 inhibitors; insulin and/or other oral diabetes medications; dexmedetomidine; ketamine; and corticosteroids, including but not limited to prednisone and/or its derivatives and synthetic analogues.

For example, onabotulinumtoxinA (Botox®) is currently used to treat migraines, cervical dystonia, blepharospasm and spasticity through injection. Botox® is a form of botulinum toxin.

Because of the drug's degradation at musculoskeletal neuronal junctions, it is believed that only a very small percentage of the injected drug actually reaches the central nervous system (CNS) tissue. It is, however, important that not too much Botox® is delivered, as the over-injection of Botox® can be lethal.

It is believed that delivering Botox® using the nasal delivery system of the present invention (if not sufficiently stable for mixed packaging, preferably mixed by a qualified physician with up to several weeks of home use using the invention) allows to ensure consistent drug delivery and to achieve an appropriate diffusion of the drug into CNS tissue, replacing the need for the multiple subcutaneous injections. In addition, the nasal administration of Botox® eliminates the risk of accidental penetration of a vessel during administration of multiple injections. Reconstitution by a physician, and mixing with the nasal spray formulation of the present invention allows for improved CNS delivery of Botox®, and may reduce migraine headache incidence and/or severity.

In addition to Botox®, other commercially available forms of botulinum toxin may be used in the formulations of the present invention.

In summary, the invention allows for the nasal administration of a drug by combining the drug with a mucosal decongestant which has a non-rebound property (such as a highly selective α-2 agonist), resulting in an improved mucoadherence and retention of the drug. This unique application system allows for everyday application of a drug with improved consistency of delivery, whether the nasal mucosa is congested or not, and improves cerebral and/or systemic drug absorption.

Thus, in one embodiment, the invention provides a method of delivering an active agent for the treatment of a systemic or cerebrovascular disease or condition comprising administering to a patient in need thereof the nasal spray composition of the invention, wherein said nasal spray composition further comprises the active agent.

The invention further provides methods of treating a disease or condition selected from the group consisting of allergies; allergic rhinitis; gastro esophageal reflux; ear infections, disseminated intravascular coagulation; allergic shock; septic shock; gastro esophageal reflux; ear infection; sinusitis; nasal congestion; migraines; headaches; cervical dystonia; blepharospasm; spasticity; Alzheimer's disease, attention deficit disorder (ADD); depression, memory loss; sleep apnea; diabetes; asthma; transient ischemic cerebrovascular ischemic attacks (TIA's); cerebrovascular accident; degenerative cerebral disorder; pneumonia; acute respiratory distress syndrome (ARDS); acute lung injury (ALI); and infantile bronchiolitis.

In preferred embodiments, the nasal spray compositions may be repeatedly administered to a patient in need thereof, for example, every eight hours, over the course of days, weeks or months, without resulting in a rebound congestion or rhinitis medicamentosa.

### Preferred Formulations of the Invention

This section of the application describes formulations of the present invention which are suitable for both treatment of nasal congestion and as delivery system to deliver additional active agents.

In one embodiment, the present invention provides compositions for nasal decongestion comprising a selective α-2 adrenergic receptor agonist having a binding affinity of 950 fold or greater for α-2 over α-1 adrenergic receptors, or a pharmaceutically acceptable salt thereof, wherein said α-2 adrenergic receptor agonist is present at a concentration from between about 0.001% and about 0.075% weight by volume.

In one embodiment, the compositions of the invention are in the form of a nasal spray. In another embodiment, the compositions of the invention are in the form of a topical drop, i.e. liquid.

In preferred embodiments, the selective α-2 adrenergic receptor agonist is selected from the group consisting of brimonidine, alpha methyl dopa, guanfacine, fadolmidine, dexmedetomidine, (+)-(S)-4-[1 -(2,3-dimethyl-phenyl)-ethyl]-1,3-dihydro-imidazole-2-thione, 1-[(imidazolidin-2-yl)imino]indazole, and mixtures of these compounds.

In the most preferred embodiment, the selective α-2 adrenergic receptor agonist is brimonidine.

Preferably, the concentration of the selective α-2 adrenergic receptor agonist is between about 0.01% and about 0.075% weight by volume, and even more preferably, between about 0.010% and about 0.035%.

In preferred embodiments, the compositions of the invention include one or more mucoadhesive additives. In a preferred embodiment, the mucoadhesive additive is selected from the group consisting of carboxymethylcellulose (CMC), hydroxypropylcellulose, carbomers and poloxamers. Preferably, the mucoadhesive additive is selected from the group consisting of a carbomer and a poloxamer or cellulose derivatives and a poloxamer. Preferably, the concentration of the poloxamer mucoadhesive additive is between about 0.5% and about 20%; more preferably, between about 5% and about 16%; and even more preferably, between about 8% and about 14%. Preferably, the concentration of the carbomer mucoadhesive additive is between about 0.05% and about 0.5%. Preferably, the concentration of the methyl cellulose derivatives, such as hydroxypropyl methylcellulose (HPMC) is between about 0.05% and about 5%, and more preferably between about 0.1% and about 0.3%.

Preferred poloxamers include, but are not limited to, Poloxamer 407 (or its trade name Pluronic® F127).

Preferably, carbomers include, but are not limited to, Carbopol® 954.

In one preferred embodiment, the concentration of poloxamer is about 12%, and the concentration of brimonidine is about 0.03%. In another preferred embodiment, the concentration of poloxamer is about 16%, and the concentration of brimonidine is about 0.025%. In another preferred embodiment, the concentration of poloxamer is about 8% and the concentration of brimonidine is about 0.035%. In another preferred embodiment, the concentration of carbomer without poloxamer is about 0.2%, the pH is between 6.0 and 7.5, and the concentration of brimonidine is about 0.035%.

In another preferred embodiment, the compositions of the invention may be administered as slow release gels, combining any two or all three of poloxamer, carbomer, and methylcellulose. In a preferred embodiment, brimonidine at 0.033% is combined with Carbopol® 954 (manufactured by Lubrizol Corporation) at 0.1% and either HPMC at 0.3%, or poloxamer at 1% to 20%, more preferably about 10%, or both. Further, polyvinyl alcohol (PVA) at 0.1% to 1%, and more preferably at 0.2% to 0.5%, and still more preferably, at 0.25% to 0.35% may be added. In some preferred embodiments, particularly when carbomers are selected as the single mucoadhesive additive it may be desired to select a surfactant for improved solubility of the alpha 2 agonist. Glycerin, or PEG and its derivatives are examples of surfactants that may be used for this purpose.

In a preferred embodiment, the invention provides a nasal spray composition comprising brimonidine tartrate, wherein said brimonidine tartrate is at a concentration from between about 0.001% and about 0.075%.

Preferably, a pH of the nasal spray composition is between about 4.0 and about 7.5; more preferably, between about 4.0 and about 6.0; and most preferably between 4.5 and 5.0.

In some preferred embodiments, the compositions of the invention may optionally comprise one or more of the following ingredients or mixtures thereof: Plasdone® K-29/32, citric acid, polysorbate 80, benzyl alcohol, propylene glycol, polyethylene glycol, microcrystalline cellulose, camphor, eucalyptol, potassium sorbate, sodium chloride and/or sterile water. An expert in the art may combine or modify the inactive ingredients listed or other inactive ingredients well known to those in the art for nasal formulations, within the concentration range for preferred super-selective α-2 agonist embodiments of the present invention to optimize preferred subjective criteria, such as comfort, and no to slight “menthol-like” sharp sensation on instillation (less than stinging); as well as objective criteria, such as onset, duration, magnitude, no or transient rebound.

The preferred concentrations of various ingredients are as follows: Plasdone® K-29/32: 2%; citric acid: 0.15%; polysorbate 80: 0.75%; benzyl alcohol: 0.5%; propylene glycol: 0.2-2%; polyethylene glycol (PEG): 2%; microcrystalline cellulose: 2% camphor and/or eucalyptol: 0.01%; potassium sorbate: 0.15%; and sodium chloride: 0.9%.

It is a goal of the present invention to provide sufficient duration of decongestant effect to allow for substantial patient benefit. A minimum of four hours of effect is desired, more preferably, six hours, and still more preferably, eight hours or more, without rebound. Virtually all formulations of the present invention provide four or more hours of effective action, and several formulations provide six or more hours. Still other preferred embodiments approach eight hours.

Table 2 lists the formulations of the invention which are most preferred for use as nasal decongestants and/or as delivery systems:

TABLE 2

| Formulation | | (PVP) | Citric | Benzyl | | |
|---|---|---|---|---|---|---|
| number | α-2 agonist | k29-32 | acid | alcohol | PS-80 | PG |
| ELDB1 | Brimonidine | 2%, 0.3 | 0.15% | 0.5%, 0.075 | 0.75%, | 1.5%, |
| | 0.045%; 4.5 | gm | | ml | 0.1125 ml | 0.225 ml |
| | ml | | | | | |
| ELDB1P1 | Brimonidine | 2%, 0.3 | 0.15% | 0.5%, 0.075 | 0.75%, | 1.5%, |
| | 0.045%; 4.5 | gm | | ml | 0.1125 ml | 0.225 ml |
| | ml | | | | | |
| ELDB2P2 | Brimonidine | 2%, 0.3 | 0.15% | 0.5%, 0.075 | 0.75%, | 1.5%, |
| | 0.05%; 5.0 | gm | | ml | 0.1125 ml | 0.225 ml |
| | ml | | | | | |
| ELDB2P3 | Brimonidine | 2%, 0.3 | 0.15% | 0.5%, 0.075 | 0.75%, | 1.5%, |
| | 0.05%; 5.0 | gm | | ml | 0.1125 ml | 0.225 ml |
| | ml | | | | | |
| ELDB3P3 | Brimonidine | 2%, 0.3 | 0.15% | 0.5%, 0.075 | 0.75%, | 1.5%, |
| | 0.035%; 3.5 | gm | | ml | 0.1125 ml | 0.225 ml |
| | ml | | | | | |

| Formulation | Avicel ® | Camphor- | Potassium | | | Poloxamer |
|---|---|---|---|---|---|---|
| number | ph 105 | eucalyptol | sorbate | pH | NaCl | 407 |
| ELDB1 | 2%, 0.3 ml | 0.01%, 0.1 ml | 0.15%, 0.0225 gm | 4.7 | 0.9%, | |
| | | | | | adjusted to | |
| | | | | | 15 cc | |
| ELDB1P1 | 2%, 0.3 ml | 0.01%, 0.1 ml | 0.15%, 0.0225 gm | 4.7 | 0.9%, | 1% |
| | | | | | adjusted to | |
| | | | | | 15 cc | |
| ELDB2P2 | 2%, 0.3 ml | 0.01%, 0.1 ml | 0.15%, 0.0225 gm | 4.7 | 0.9%, | 4% |
| | | | | | adjusted to | |
| | | | | | 15 cc | |
| ELDB2P3 | 2%, 0.3 ml | 0.01%, 0.1 ml | 0.15%, 0.0225 gm | 4.7 | 0.9%, | 8% |
| | | | | | adjusted to | |
| | | | | | 15 cc | |
| ELDB3P3 | 2%, 0.3 ml | 0.01%, 0.1 ml | 0.15%, 0.0225 gm | 4.5 | 0.9%, | 8% |
| | | | | | adjusted to | |
| | | | | | 15 cc | |

Avicel ® ph 105 (manufactured by FMC Corporation) is a microcrystalline cellulose;
(PVP) k29-32 is polyvinylpyrrolidone (Plasdone ®, manufactured by International
Specialty Products) k29-32;
P-80 is polysorbate 80;
PG is propylene glycol;
CA is citric acid; and
BA is benzyl alcohol.

In another preferred embodiment (NX035P8), the concentrations of various ingredients are as follows:

| | | |
|---|---|---|
| Brimonidine Tartrate 0.15% | 3.5 | ml |
| Plasdone ® k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.0225 | gm |
| Poloxamer 407 20% | 6 | ml |
| Preserved Sodium Chloride Irrigation | qs 15 | ml |
| pH < 5.0, about 4.5 | | |

The final brimonidine concentration is 0.035%, and the final poloxamer concentration is 8%.

In another preferred embodiment (NX01P16), the concentrations of various ingredients are as follows:
Brimonidine tartrate 0.012% (0.005%-0.015% preferred range)
PEG 1450 2%
PG 2%
Potassium sorbate 0.10%
Poloxamer 407 16%
Sterile water
Camphor/Eucalyptol Mix 0.66%
pH about 4.5-6.0

In another preferred embodiment (NX030 WP12), the invention provides:

1- 15 ml Nasal Spray

| | | |
|---|---|---|
| Brimonidine Tartrate 0.15% Ophthalmic Solution | 3.0 | ml |
| PEG 1450 | 0.375 | gm |
| Propylene Glycol, USP | 0.3 | ml |
| Poloxamer 407 25% | 7.2 | ml |
| Camphor/Eucalyptol Mix | 0.1 | ml |
| Potassium Sorbate, NF | 0.015 | gm |
| Sterile Water | qs 15 | ml |

In another preferred embodiment (NX010WP16), the invention provides:

1- 15 ml Nasal Spray

| | | |
|---|---|---|
| Brimonidine Tartrate 0.15% Ophthalmic Solution | 0.75 | ml |
| PEG 1450 | 0.375 | gm |
| Propylene Glycol, USP | 0.3 | ml |
| Poloxamer 407 25% | 9.6 | ml |
| Camphor/Eucalyptol Mix | 0.1 | ml |
| Potassium Sorbate, NF | 0.015 | gm |
| Sterile Water | qs 15 | ml |

In another preferred embodiment (NX025WC1PVA3HPMC3) the invention provides:
Brimonidine Tartrate 0.025%
Carbopol 954 0.1%
Poloxamer 407 16%
PVA 0.3%
HPMC 0.3%
Sterile water
Potassium Sorbate, NF 0.10%
pH 5.6

In another preferred embodiment (NX033P11WC1PVA3HPMC3) the invention provides:
Brimonidine Tartrate 0.033%
Carbopol 954 0.1%
Poloxamer 407 11.2% (8 to 11%)
PVA 0.3%
HPMC 0.3%
Sterile water
Potassium Sorbate, NF 0.10%
pH 4.6

Other preferred embodiments include:

| NX020WP15PVA2HPMC2MC2 | | |
|---|---|---|
| Brimonidine 0.022% | | |
| PEG 1450 2.5% | | |
| Propylene Glycol 2% | | |
| Poloxamer 15% | | |
| Camphor-eucalyptol 0.01% | | |
| Potassium sorbate 0.1% | | |
| PVA 0.3% | | |
| HPMC 0.3% | | |
| sterile water | | |
| Microcrystalline Cellulose 0.2 gm | | |
| If this is too thick to spray: | | |
| 1) first decrease HPMC to 0.2% or 0.1% or | | |
| eliminate altogether; | | |
| 2) if this is not enough, then decrease | | |
| microcrystalline cellulose as needed. | | |
| NX020WP15PVA2.8HPMC2.8 | | |
| Brimonidine 0.020% | | |
| PEG 1450 2.5% | | |
| Propylene Glycol 2% | | |
| Poloxamer 15% | | |
| Camphor-eucalyptol 0.01% | | |
| Potassium sorbate 0.1% | | |
| PVA 0.28% | | |
| HPMC 0.28% | | |
| sterile water | | |
| NX022P16PVA3HPMC3 C1 | | |
| Brimonidine 0.020% | | |
| PEG 1450 2.5% | | |
| Propylene Glycol 2% | | |
| Poloxamer 16% | | |
| Camphor-eucalyptol 0.01% | | |
| Potassium sorbate 0.1% | | |
| PVA 0.3% | | |
| HPMC 0.3% | | |
| Carbopol 0.1% | | |
| sterile water | | |
| NX022P15PVA3HPMC3C05 | | |
| Brimonidine 0.020% | | |
| PEG 1450 2.5% | | |
| Propylene Glycol 2% | | |
| Poloxamer 15% | | |
| Camphor-eucalyptol 0.01% | | |
| Potassium sorbate 0.1% | | |
| PVA 0.3% | | |
| HPMC 0.3% | | |
| Carbopol 0.05% | | |
| sterile water | | |
| NX035P8C1 | | |
| Brimonidine Tartrate 0.15% | 3.5 | ml |
| Plasdone ® k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.0225 | gm |
| Poloxamer 407 20% | 6 | ml |
| Carbopol 934P | 0.1% | |
| Preserved Sodium Chloride Irrigation | qs 15 | ml |
| NX030P8C1 | | |
| Brimonidine Tartrate | 3.0 | ml |
| Plasdone ® k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.0225 | gm |
| Poloxamer 407 20% | 6 | ml |
| Carbopol 934P | 0.1% | |
| Preserved Sodium Chloride Irrigation | qs 15 | ml |
| NX035P8PVA05HPMC05 | | |
| Brimonidine Tartrate | 3.5 | ml |
| Plasdone ® k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.0225 | gm |
| Poloxamer 407 20% | 6 | ml |
| PVA | 0.05% | |
| HPMC | 0.05% | |
| Preserved Sodium Chloride Irrigation | qs 15 | ml |
| NX035P10PVA05HPMC05 | | |
| Brimonidine Tartrate | 3.5 | ml |
| Plasdone ® k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.015 | gm |
| Poloxamer 407 20% | 7.5 | ml |
| PVA | 0.05% | |
| HPMC | 0.05% | |
| Preserved Sodium Chloride Irrigation 0.45% | qs 15 | ml |
| NX030P10PVA05HPMC05C1 | | |
| Brimonidine Tartrate 0.15% | 3.0 | ml |
| Plasdone k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.1 | gm |
| Camphor/Eucalyptol Mix | 0.1 | ml |
| Potassium Sorbate, NF | 0.015 | gm |
| Poloxamer 407 20% diluted | 10% | |
| PVA | 0.05% | |
| PMC | 0.05% | |
| Preserved Sodium Chloride Irrigation 0.45% | qs 15 | ml |
| NX035P10 | | |
| Brimonidine Tartrate 0.15% | 3.5 | ml |
| Plasdone k-29/32, USP | 0.3 | gm |
| Citric Acid, USP Anhydrous fine granular | 0.0225 | gm |
| Polysorbate 80, NF | 0.1125 | ml |
| Benzyl Alcohol, NF | 0.075 | ml |
| Propylene Glycol, USP | 0.225 | ml |
| Microcrystalline cellulose (PH-105) | 0.3 | gm |
| Camphor/Eucalyptol Mix | 0.1 | ml |
| Potassium Sorbate, NF | 0.10% | |
| Poloxamer 407 20% diluted | 10% | |
| Preserved Sodium Chloride Irrigation 0.45% | qs 15 | ml |

In one embodiment, Poloxamer 407 25% gel used to create the provided formulations is as follows:

| | | |
|---|---|---|
| Poloxamer 407, NF | 25 | gm |
| Sterile Water | qs 100 | ml |

In one embodiment, Camphor/Eucalyptol mix is as follows:

| | | |
|---|---|---|
| Camphor, USP Synthetic | 0.15 | gm |
| Eucalyptol, USP | 0.1625 | ml |
| Preserved Sodium Chloride Irrigation | qs 10 | ml |

The compositions of the invention may also comprise a solubility stabilizer which preferably contains an anionic component, such as peroxide class preservatives. The solubility stabilizer allows one to achieve greater penetration of lipophilic membranes, such as those present at the vascular endothelial surface. In a preferred embodiment, the solubility stabilizer comprises a stabilized oxychloro complex, chlorite, and sodium perborate. Other additives, such as ethylenediaminetetraacetic acid (EDTA) or citric acid, may be used where increased mucosal penetration is desired. Potassium sorbate may be used as a preservative to replace benzalkonium chloride (BAK), with known rebound inducing toxicity, or the formulations may be delivered in disposable unit dose nasal spray delivery devices.

In yet another embodiment, the compositions of the present invention may comprise nitrous oxide inhibitors. In a preferred embodiment, the nitrous oxide inhibitors are selected from the group consisting of L-NAME (L-N^{G}-Nitroarginine methyl ester), L-NIL (N6-(1-Iminoethyl)-L-lysine dihydrochloride), L-NIO (N5-(1-Iminoethyl)-L-ornithine dihydrochloride), and L-canavine, or combinations thereof. Preferably, concentration of the nitrous oxide inhibitors is between about 0.005% and about 0.5% weight by volume.

The compositions of the invention may also include additional components, which include, but are not limited to, preservatives, delivery vehicles, tonicity adjustors, buffers, pH adjustors, antioxidants, and water.

The preservatives include, but are not limited to, potassium sorbate, peroxide and peroxide derivatives, benzalkonium chloride, chlorobutanol, thimerosal, phenylmercuric acetate, or phenylmercuric nitrate. Of these, potassium sorbate is used in preferred embodiments. Vehicles useful in a topical composition include, but are not limited to, polyvinyl alcohol, glycerin, povidone, hydroxypropyl methyl cellulose, poloxamers, carboxymethyl cellulose, hydroxyethyl cellulose and purified water. It is also possible to use sterile water or a physiological saline solution as a major vehicle.

A tonicity adjustor also can be included, if desired, in a topical composition of the invention. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare topical compositions of the invention. Such buffers include, but are not limited to, acetate buffers, citrate buffers, phosphate buffers and borate buffers. It is understood that acids or bases can be used to adjust the pH of the composition as needed. Topically acceptable antioxidants useful in preparing a topical composition include, yet are not limited to, sodium metabisulfite, sodium thiosulfate, acetylcysteine, butylated hydroxyanisole and butylated hydroxytoiuene.

In one embodiment, the provided composition is an aerosolized composition. It is within a skill in the art to prepare aerosolized compositions of the present invention. The aerosolized compositions of the present invention are generally delivered via an inhaler, jet nebulizer, or ultrasonic nebulizer which is able to produce aerosol particles with size of between about 1 and 10 μm.

To make the topical compositions of the present invention, one can simply dilute, using methods known in the art, more concentrated solutions of selective α-2 agonists. The precise method of carrying out the dilutions is not critical. Any commonly used diluents, including preservatives described above in the application, suitable for topical solutions can be used. Addition of poloxamer may be performed by initially dissolving using cold technique and overnight refrigeration well known to experts in the art.

Proper dosages of the compositions of the present invention are concentration-dependent. To determine the specific dose for a particular patient, a skilled artisan would have to take into account kinetics and absorption characteristics of the particular highly selective α-2 adrenergic receptor agonist.

The following Examples are provided solely for illustrative purposes and are not meant to limit the invention in any way.

### EXAMPLES

### Example 1

### Manufacturing ELDB2P3 Formulation

While there is no specific limitation on the method of manufacturing formulations of the invention, ELDB2P3 formulation was created as follows:

Plasdone® (PVP k29-32), citric acid, microcrystalline cellulose, potassium sorbate were dissolved in 45 ml of preserved NaCl irrigation. To this mixture, polysorbate 80, benzyl alcohol, propylene glycol, and camphor-eucalyptol mix were added. Poloxamer 407 was stirred in for at least one hour, and refrigerated overnight to totally dissolve. Then, the active ingredient (brimonidine 0.05%) was added.

The resultant formulation had no undesirable aftertaste and no stinging. It provided an almost immediate onset of less than 10 seconds. It created a long duration high magnitude decongestant effect without pharyngeal dryness. As there is no preservative, and the pH (about 4.5) is highly acidic, it is believed that the formulation can be used regularly without the rebound congestion.

### Example 2

### Comparison of the Formulations of the Invention

### Experimental Design

To determine which compositions of the invention are the most optimal, two representative formulations were tested. The first formulation, NX040P2, included brimonidine at 0.040% and poloxamer at 2%, while the second formulation, NX050P4, included brimonidine at 0.05% and poloxamer at 4%.

Both formulations were applied three times daily (separately from each other and on different days. The formulations were applied as follows. First, the bottle was shaken well for about 10 seconds. The same nasal spray bottle with about 0.10 - 0.15 cc per spray was then pumped twice into air to ensure a filled chamber, and then a single spray was administered to each nostril. This was repeated two more times at 8 hour intervals, with results recorded after each administration:

### Results

The results of this experiment are described in Table 3 below.

TABLE 3

| | | | | | Congestion | Side Effects | | |
|---|---|---|---|---|---|---|---|---|
| | | Noticeable | Magnitude (at peak) | | After Use | (scale from 0 to 4) | | |
| TID Test (three times a | | Onset | (scale | Duration | (scale from | | | Dry |
| day) | | (min) | from 0 to 4) | (hrs) | 0 to 4) | Sting | Sedation | mouth |
| NX040P2 | 1^{st} | 10 | 4 | 5 | 0 | 0 | 0 | 0 |
| | use | | | | | | | |
| | 2^{nd} | 10 | 4 | 6.5 | 0 | 0 | 0 | 0 |
| | use | | | | | | | |
| | 3^{rd} | 10 | 4 | 6.0 | 0 | 0 | 0 | 0 |
| | use | | | | | | | |
| NX050P4 | 1^{st} | 5 | 4 | 8 | 0 | 1 | 0 | 0 |
| | use | | | | | | | |
| | 2^{nd} | 5 | 4 | 6.5 | 1 | 1 | 0 | 1 |
| | use | | | | | | | |
| | 3^{rd} | 5 | 4 | 6 | 2 | 1 | 1 | 1 |
| | use | | | | | | | |

It was discovered that NX050P4, but not NX040P2 resulted in about two hours of post-instillation nasal congestion after about 8 hours of effect. NX050P4 also caused noticeable pharyngeal dryness, while with the use of NX040P2, pharyngeal dryness was barely detectable. NX040P2 had a slightly slower onset than NX050P4, but a similar duration of a bit more than 5 hours of action.

### Example 3

A range of combinations of brimonidine with various mucoadhesives and inactive ingredient combinations were studied for
1) onset of nasal decongestion on a 1(least) to 4 (most) scale;
2) magnitude of decongestion of turbinates (1-4);
3) duration of decongestion (hrs)
4) pharyngeal dryness;
5) sedation;
6) stinging sensation in nostrils;
7) post instillation congestion of <2 hours (no cases occurred greater than 2 hours, (yes or no))

### Experimental Design

Each formulation was administered to a test subject with partial turbinate blockage—air patency but labored breathing required to get air through nostrils. Following loss of effect, a second instillation was repeated and the results were recorded. No washout period was observed. Most formulations were tested more than once and the results were collated. At all times, a return to normal baseline was observed the following day, provided single use or twice daily daytime use. Table 4 describes the components of the formulations and the results achieved with their use.

TABLE 4
FORMULATION TEST GRID

| | Brimonidine | Poloxamer | | |
|---|---|---|---|---|
| | Concentration | Concentration | Onset | Magnitude of |
| Formulation | (%) | (%) | (min) | Effect |
| NX025P0 | 0.025 | 0 | 5-10 | 2.00 |
| NX035P0 | 0.035 | 0 | 5-10 | 2.00 |
| NX045P0 | 0.045 | 0 | 3 | 2.50 |
| NX050P0 | 0.050 | 0 | 2-3 | 3.00 |
| NX045P1 | 0.045 | 1 | 2 | 3.75 |
| NX050P1 | 0.050 | 1 | 2 | 4.00 |
| NX050P2 | 0.050 | 2 | 2 | 4.00 |
| NX035P2 | 0.035 | 2 | 2 | 4.00 |
| NX035P3 | 0.035 | 3 | 2 | 4.00 |
| NX050P4 | 0.050 | 4 | 1 | 4.00 |
| NX025P6 | 0.025 | 6 | 5 | 3.00 |
| NX035P6 | 0.035 | 6 | 5 | 4.00 |
| NX050P6 | 0.050 | 6 | 5 | 4.00 |
| NX025P8 | 0.025 | 8 | 5-10 | 3.00 |
| NX050P8 | 0.050 | 8 | 1 | 4.00 |
| NX035P12 | 0.035 | 12 | 5-10 | 4.00 |
| NX030P12 | 0.030 | 12 | 5-10 | 4.00 |
| NX030P16 | 0.030 | 16 | 1 | 4.00 |
| NX025P16 | 0.025 | 16 | 2-3 | 4.00 |
| NX020P16 | 0.020 | 16 | 2-3 | 3.00 |
| NX035P16 | 0.035 | 16 | 1 | 4.00 |
| NX025P16 | 0.025 | 16 | 2 | 4.00 |
| NX015P16 | 0.015 | 16 | 3-5 | 4.00 |
| NX025WP16VA3HPMC3 | 0.025 | 16 | 3 | 4.00 |
| NX030WC1PVA3HPMC3 | 0.030 | 16 | 3-5 | 4.00 |

| | | | | | | | Comment |
|---|---|---|---|---|---|---|---|
| | Side Effects | | | | Post | | +++++ = |
| | Duration | Dryness | Sedation | Stinging | use ≦2 h | Other Key | Most |
| Formulation | hrs. | 0-4 | 0-4 | 0-4 | rebound | Inactives | preferred |
| NX025P0 | 2.50 | 0 | 0 | 0 | no | PVP, PG, | + |
| | | | | | | Avicel ®, | |
| | | | | | | poly80, | |
| | | | | | | benzyl | |
| | | | | | | alcohol, citric | |
| | | | | | | acid | |
| NX035P0 | 3.00 | 0 | 0 | 0 | no | PVP, PG, | ++ |
| | | | | | | Avicel ®, | |
| | | | | | | poly80, | |
| | | | | | | benzyl | |
| | | | | | | alcohol, citric | |
| | | | | | | acid | |
| NX045P0 | 3.50 | 0 | 0 | 0 | yes | PVP, PG, | ++ |
| | | | | | | Avicel ®, | |
| | | | | | | poly80, | |
| | | | | | | benzyl | |
| | | | | | | alcohol, citric | |
| | | | | | | acid | |
| NX050P0 | 4.00 | 1 | 0 | 1 | yes | PVP, PG, | + |
| | | | | | | Avicel ®, | |
| | | | | | | poly80, | |
| | | | | | | benzyl | |
| | | | | | | alcohol, citric | |
| | | | | | | acid | |
| NX045P1 | 5.00 | 1 | 0 | 1 | yes | PVP, PG, | +++ |
| | | | | | | Avicel ®, | |
| | | | | | | poly80, | |
| | | | | | | benzyl | |
| | | | | | | alcohol, citric | |
| | | | | | | acid | |
| NX050P1 | 6.00 | 1 | 0 | 1 | yes | PVP, pg, | ++ |
| | | | | | | Avicel ®, | |
| | | | | | | poly80, | |
| | | | | | | benzyl | |
| | | | | | | alcohol, citric | |
| | | | | | | acid | |
| NX050P2 | 7.00 | 1 | 0 | 0.5 | yes | PVP, PG, | +++ |
| | | | | | | Avicel ®, | |
| | | | | | | poly80, | |
| | | | | | | benzyl | |
| | | | | | | alcohol, citric | |
| | | | | | | acid | |
| NX035P2 | 7.00 | 0.5 | 0 | 0 | no | PVP, PG, | ++++ |
| | | | | | | Avicel ®, | |
| | | | | | | poly80, | |
| | | | | | | benzyl | |
| | | | | | | alcohol, citric | |
| | | | | | | acid | |
| NX035P3 | 5.00 | 0.5 | 0 | 0 | no | PVP, PG, | ++++ |
| | | | | | | Avicel ®, | |
| | | | | | | poly80, | |
| | | | | | | benzyl | |
| | | | | | | alcohol, citric | |
| | | | | | | acid | |
| NX050P4 | 5.00 | 1 | 0 | 1 | yes | PVP, PG, | + |
| | | | | | | Avicel ®, | |
| | | | | | | poly80, | |
| | | | | | | benzyl | |
| | | | | | | alcohol, citric | |
| | | | | | | acid | |
| NX025P6 | 5.00 | 0 | 0 | 0 | no | PVP, PG, | +++ |
| | | | | | | Avicel ®, | |
| | | | | | | poly80, | |
| | | | | | | benzyl | |
| | | | | | | alcohol, citric | |
| | | | | | | acid | |
| NX035P6 | 6.00 | 0 | 0 | 0 | no | PVP, PG, | +++ |
| | | | | | | Avicel ®, | |
| | | | | | | poly80, | |
| | | | | | | benzyl | |
| | | | | | | alcohol, citric | |
| | | | | | | acid | |
| NX050P6 | 5.00 | 1.5 | 0 | 0.75 | yes | PVP, PG, | + |
| | | | | | | Avicel ®, | |
| | | | | | | poly80, | |
| | | | | | | benzyl | |
| | | | | | | alcohol, citric | |
| | | | | | | acid | |
| NX025P8 | 5.00 | 0 | 0 | 0 | no | PVP, PG, | ++ |
| | | | | | | Avicel ®, | |
| | | | | | | poly80, | |
| | | | | | | benzyl | |
| | | | | | | alcohol, citric | |
| | | | | | | acid | |
| NX035P8 | 6.00 | 0 | 0 | 0 | no | PVP, PG, | ++++½* |
| | | | | | | Avicel ®, | |
| | | | | | | poly80, | |
| | | | | | | benzyl | |
| | | | | | | alcohol, citric | |
| | | | | | | acid | |
| NX050P8 | 5.00 | 1.5 | 0 | 1 | yes | PEG, PG, | + |
| | | | | | | sterile water, | |
| | | | | | | sorbate | |
| NX035P12 | 6.00 | 0 | 0 | 1 | yes | PEG, PG, | ++ |
| | | | | | | sterile water, | |
| | | | | | | sorbate | |
| NX030P12 | 7.00 | 0 | 0 | 0.5 | no | PEG, PG, | +++ |
| | | | | | | sterile water, | |
| | | | | | | sorbate | |
| NX030P16 | 5.00 | 0.5 | 0 | 1.5 | yes | PEG, PG, | ++ |
| | | | | | | sterile water, | |
| | | | | | | sorbate | |
| NX025P16 | 6.00 | 0 | 0 | 1.5 | yes | PEG, PG, | +++* |
| | | | | | | sterile water, | |
| | | | | | | sorbate | |
| NX020P16 | 6.00 | 0 | 0 | 0 | no | PEG, PG, | ++++ |
| | | | | | | sterile water, | |
| | | | | | | sorbate | |
| NX035P16 | 3.50 | 0 | 0 | 2 | yes | PEG, PG, | +* |
| | | | | | | sterile water, | |
| | | | | | | sorbate | |
| NX025P16 | 4.00 | 0 | 0 | 1 | yes | PEG, PG, | ++ |
| | | | | | | sterile water, | |
| | | | | | | sorbate | |
| NX025WP15V | 7.50 | 0 | 0 | 0.5 | yes | PVA, HPMC, | ++++* |
| A3HPMC3 | | | | | | sterile water, | |
| | | | | | | sorbate | |
| NX030WC1P | 3.50 | 0.5 | 0 | 0 | yes | Carbomer, | +++ |
| VA3HPMC3 | | | | | | PVA, HPMC | |
| NX015WP16P | 5.50 | 0 | 0 | 0 | no | PVA, HPMC, | +++* |
| VA3HPMC3 | | | | | | sterile water, | |
| | | | | | | sorbate | |
| NX020WP15P | 7.50 | 0 | 0 | 0 | no | PVA, HPMC, | +++++* |
| VA28HPMC28 | | | | | | sterile water, | |
| | | | | | | sorbate | |
| NX020WP15P | 11.50 | 0 | 0.25 | 0 | no | PVA, HPMC, | +++++** |
| VA28HPMC2 | | | | | | sterile water, | |
| | | | | | | sorbate | |

Avicel ® (manufactured by FMC Corporation) is a microcrystalline cellulose;
PVP is polyvinylpyrrolidone (Plasdone ®, manufactured by International Specialty Products) k29-32;
poly80 is polysorbate 80;
PEG is polyethylene glycol; and
PG is propylene glycol;
*formulation administered through 3 sprays per nostril, 1-2 minutes apart
**1-2 drops per eye brimonidine 0.025% ophthalmic administered at time of nasal instillation

### Example 4

### Synergy Between Ophthalmic Administration and Nasal Administration of α-2 Agonists

The main purpose of this experiment was to determine whether ophthalmic co-administration of brimonidine 0.025% increases the duration of the effect of nasal administration of a preferred composition of the present invention. In addition, the experiment was to determine whether multiple consecutive (1-3 hours apart) instillations of the compositions of the invention for about 48 hours cause any rebound congestion.

### Experimental Design

A preferred composition of the invention, NX020P15PVA3HPMC3, was administered nasally, with repeat administration 1-3 hours after return to baseline five consecutive times. On the third and fifth instillation, ophthalmic dosing was added.

### Results

| NX020P15PVA3HPMC3 | Brim 0.025% | | Duration |
|---|---|---|---|
| (Nasal) | Ophthalmic | Rebound | (hrs) |
| 1^{st} instillation | − | No | 7.25 |
| 2^{nd} instillation | − | No | 7.50 |
| 3^{rd} instillation | + | No | 11.50 |
| 4^{th} instillation | − | No | 7.45 |
| 5^{th} instillation | + | No | 11.25 |

| | |
|---|---|
| Total Hours of Effect in a 54 hour time block: | 44.95 |

Co-administration of the active ingredient of the present invention via ophthalmic and nasal delivery (3^{rd} and 5^{th} nasal instillations) vs. nasal delivery alone (1^{st}, 2^{nd}, and 4^{th} instillations) resulted in a substantial increased duration of action to a mean of about 11.37 hours vs. about 7.39 hours for nasal dosing alone.

Five consecutive nasal applications, with only slight lag time between doses and ophthalmic administration on 3^{rd} and 5^{th} instillation, resulted in effective decongestant action for about 83% of a 54 hour time frame without rebound. There were no adverse effects on a patient.

Coincidentally, on the 3^{rd} instillation topical ophthalmic brimonidine 0.025% was administered to remove baseline eye redness in a subject, and an 11.5 hour duration was unexpectedly found. To confirm that this surprising and completely unexpected extended duration was not spurious or unrelated to the ophthalmic delivery, a fourth instillation (without ophthalmic brimonidine) followed by a fifth instillation (with ophthalmic brimonidine) were administered. The fourth instillation had no extended duration effect, while the fifth instillation had a similar extended duration effect.

What is claimed is:

1. A nasal spray composition comprising a selective α-2 adrenergic receptor agonist having a binding affinity of 900 fold or greater for α-2 over α-1 adrenergic receptors, or a pharmaceutically acceptable salt thereof, wherein said α-2 adrenergic receptor agonist is present at a concentration from between about 0.001% to about 0.075% weight by volume.

2. The nasal spray composition of claim 1, wherein said selective α-2 adrenergic receptor agonist is selected from the group consisting of, brimonidine, alpha methyl dopa, guanfacine, fadolmidine, dexmedetomidine, (+)-(S)-4-[1-(2,3-dimethyl-phenyl)-ethyl]-1,3-dihydro-imidazole-2-thione, 1-[(imidazolidin-2-yi)imino]indazole, and mixtures of these compounds.

3. The nasal spray composition of claim 1, wherein said selective α-2 adrenergic receptor agonist is brimonidine.

4. The nasal spray composition of claim 3, wherein said brimonidine is at a concentration from between about 0.001% and about 0.05%.

5. The nasal spray composition of claim 1, further comprising a mucoadhesive additive.

6. The nasal spray composition of claim 5, wherein said mucoadhesive additive is selected from the group consisting of carboxymethylcellulose, hydroxypropylcellulose, other cellulose derivatives, guar gum, xanthan gum, carbomers, poloxamers, chondroitin sulfate and mixtures thereof.

7. The nasal spray composition of claim 6, wherein said mucoadhesive additive is Poloxamer 407.

8. A method of treatment of nasal congestion comprising administering to a patient in need thereof a pharmaceutical composition comprising: a selective α-2 adrenergic receptor agonist having a binding affinity of 900 fold or greater for α-2 over α-1 adrenergic receptors, or a pharmaceutically acceptable salt thereof, wherein said α-2 adrenergic receptor agonist is present at a concentration from between about 0.001% to about 0.075% weight by volume.

9. The method of claim 8, wherein said composition is a nasal spray.

10. The method of claim 8, further comprising topically ophthalmically administering to said patient said selective α-2 adrenergic receptor agonist as an eye drop.

11. The method of claim 10, where the concentration of said selective α-2 agonist administered as an eye drop is between about 0.005% and about 0.050% weight by volume.

12. The method of claim 8, wherein said composition further comprises one or more mucoadhesive additives.

13. The method of claim 12, wherein said mucoadhesive additive is selected from the group consisting of carboxymethylcellulose, hydroxypropylcellulose, other cellulose derivatives, guar gum, xanthan gum, carbomers, poloxamers. chondroitin sulfate and mixtures thereof.

14. A method of delivering an active agent for the treatment of a systemic or cerebrovascular disease or condition comprising administering to a patient in need thereof a pharmaceutical composition comprising: 1) a selective α-2 adrenergic receptor agonist having a binding affinity of 900 fold or greater for α-2 over α-1 adrenergic receptors, or a pharmaceutically acceptable salt thereof, wherein said α-2 adrenergic receptor agonist is present at a concentration from between about 0.001% to about 0.075% weight by volume; and 2) an active agent for the treatment of said systemic or cerebrovascular disease or condition.

15. The method of claim 14, wherein said pharmaceutical composition further comprises a mucoadhesive additive.

16. The method of claim 15, wherein said mucoadhesive additive is selected from the group consisting of carboxymethylcellulose, hydroxypropylcellulose, other cellulose derivatives, guar gum, xanthan gum, carbomers, poloxamers, chondroitin sulfate and mixtures thereof.

17. The method of claim 14, wherein said systemic or cerebrovascular disease or condition is selected from the group consisting of allergies; allergic rhinitis; disseminated intravascular coagulation; allergic shock; septic shock; gastro esophageal reflux; ear infection; sinusitis; nasal congestion; migraines; headaches; cervical dystonia; blepharospasm; spasticity; Alzheimer's disease, attention deficit disorder (ADD); depression, memory loss; sleep apnea; diabetes; asthma; transient ischemic cerebrovascular ischemic attacks (TIA's); cerebrovascular accident; degenerative cerebral disorder; pneumonia; acute respiratory distress syndrome (ARDS); acute lung injury (ALI); and infantile bronchiolitis.

18. The method of claim 14, wherein said active agent is selected from the group consisting of onabotulinumtoxinA, another botulinum polymer, ketorolac tromethamine, sumatriptan, dihydroergotamine, indomethacin, ibuprofen, sulindac sulfide, meclofenamic acid, flurbiprofen, hydroxycholoroquine, insulin, sulfonylureas, other oral hypoglycemics, dexmedetomidine, ketamine, prednisone, and combinations thereof.

19. A nasal spray composition comprising brimonidine, wherein said brimonidine is at a concentration from between about 0.001% and about 0.075%.

20. The nasal spray composition of claim 19, wherein pH of said composition is between about 4.0 and about 7.5.

21. The nasal spray composition of claim 19 further comprising one or more mucoadhesive additives.

22. The nasal spray composition of claim 21, wherein said mucoadhesive additive is selected from the group consisting of a carbomer and a poloxamer.

23. The nasal spray composition of claim 21, wherein said poloxamer is Poloxamer 407,

24. The nasal spray composition of claim 23, wherein said poloxamer is at a concentration from between about 2% and about 20%.

25. The nasal spray composition of claim 22, wherein said carbomer is Carbopol® 954.

26. The nasal spray composition of claim 25 where said carbomer is at a concentration from between 0.05% and 0.5%.

27. The nasal spray composition of claim 19 further comprising one or more ingredients selected from the group consisting of poloxamer, carbomer, hydroxypropylmethylcellulose, polyvinyl alcohol, sterile water, camphor, eucalyptol, and potassium sorbate.

28. A method of treating a headache comprising administering to a patient in need thereof a therapeutically effective amount of the nasal spray composition of claim 19.

29. A method of treating atopy comprising administering to a patient in need thereof a therapeutically effective amount of the nasal spray composition of claim 19 and an allergen.

30. A method of treating rhinitis medicamentosa comprising administering to a patient in need thereof a therapeutically effective amount of the nasal spray composition of claim 19.

31. A method of treating a disease or condition characterized by loss of intravascular volume due to vascular leakage comprising administering to a patient in need thereof a therapeutically effective amount of the nasal spray composition of claim 19.

32. The method of claim 31, further comprising administering to said patient dexmedetomidine and/or ketamine.
